(19) United States
(12) Patent Application Publication
(10) Pub. No.: US 2025/0217442 A1
(43) Pub. Date: Jul. 3, 2025

(54) SYSTEMS AND METHODS FOR DETECTING UNNECESSARY RESOURCE RE-UTILIZATION

(71) Applicant: Optum, Inc., Minnetonka, MN (US)

(72) Inventors: Yao ZHANG, La Canada Flintridge, CA (US); James M. DOLSTAD, Leavenworth, WA (US); Alice LI, New York, NY (US); Aparna PHILKANA, Naples, FL (US); Maxine GOLDSMITH, West Hartford, CT (US); Rajiv ARYA, Skillman, NJ (US)

(21) Appl. No.: 18/400,043

(22) Filed: Dec. 29, 2023

(51) Int. Cl. G06F 17/18; G06F 16/2457; G16H 40/20

(52) U.S. Cl. CPC G06F 17/18; G06F 16/24573; G16H 40/20

(57) ABSTRACT

Systems and methods are disclosed for detecting unnecessary resource re-utilization. A method includes receiving a first data object, the first data object including an entity data set containing a plurality of entities; a first data set including request data associated with the plurality of entities; an event data set; and a plurality of data sets associated with one or more performance metrics. The method further includes generating an entity data object for each of the plurality of entities and applying a machine-learning model to the entity data objects generated for the plurality of entities. The method further includes determining a prediction indicator for each entity of the plurality of entities, generating a re-utilization offset data object for each of the plurality of entities, and causing the re-utilization offset data object for each entity to be displayed on a Graphical User Interface (GUI).

### TECHNICAL FIELD

The present disclosure generally relates to the field of data analytics. In particular, the present disclosure relates to systems and methods for scenario modeling and predicting resource re-utilization based on various data sources, to generate interventions that increase efficiency of resource utilization.

### BACKGROUND

Inefficient or unnecessary resource re-utilization is not only costly for organizations but often indicates suboptimal management of or offsite support for entities associated with the organizations. A variety of techniques have been implemented to reduce avoidable resource re-utilizations, including enhanced entity education, improved post-utilization or offsite planning, follow-up measures, and coordination with entity management services. However, these techniques suffer from one or more issues and may be improved in one or more ways.

For instance, current techniques often struggle to effectively identify entities at high risk of avoidable resource re-utilizations. While entity education and post-utilization or offsite planning are valuable, they may not be sufficiently tailored to individual entities' needs and circumstances. Follow-up measures can be effective, but rely heavily on entities' ability and willingness to participate, which can be influenced by numerous factors. Coordination with entity management services is crucial, yet often hampered by systemic communication barriers.

Therefore, there is a need for a more sophisticated and accurate approach to predicting and mitigating unnecessary, or avoidable, resource re-utilizations.

This disclosure is directed to addressing the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure addresses the technical problem(s) described above or elsewhere in the present disclosure and improves the state of conventional resource management techniques.

In some aspects, the techniques described herein relate to a computer-implemented method, the method including: receiving, by one or more processors, a first data object, the first data object including: an entity data set containing a plurality of entities; a first data set including request data associated with the plurality of entities; an event data set; and a plurality of data sets associated with one or more performance metrics; generating, by the one or more processors, based on at least one of the entity data set, the first data set, or the event data set, an entity data object for each of the plurality of entities; applying, by the one or more processors, a machine-learning model to the entity data objects generated for the plurality of entities, the machine-learning model trained to identify a correlation between the entity data object for each of the plurality of entities and a probability of re-utilization of one or more resources; determining, by the one or more processors, based on the application of the machine-learning model to the entity data objects, a prediction indicator for each entity of the plurality of entities; generating, by the one or more processors, a re-utilization offset data object for each of the plurality of entities, the re-utilization offset data object based on the prediction indicator determined for the entity; and causing, by the one or more processors, one or more of the re-utilization offset data objects generated for the plurality of entities to be displayed on a Graphical User Interface (GUI).

In some aspects, the techniques described herein relate to a system including memory and one or more processors communicatively coupled to the memory, the one or more processors configured to: receive, a first data object, the first data object including: an entity data set containing a plurality of entities; a first data set including request data associated with the plurality of entities; an event data set; and a plurality of data sets associated with one or more performance metrics; generate, based on at least one of the entity data set, the first data set, or the event data set, an entity data object for each of the plurality of entities; apply a machine-learning model to the entity data objects generated for the plurality of entities, the machine-learning model trained to identify a correlation between the entity data object for each of the plurality of entities and a probability of re-utilization of one or more resources; determine, based on the application of the machine-learning model to the entity data objects, a prediction indicator for each entity of the plurality of entities; generate, a re-utilization offset data object for each of the plurality of entities, the re-utilization offset data object based on the prediction indicator determined for the entity; and cause one or more of the re-utilization offset data objects generated for the plurality of entities to be displayed on a Graphical User Interface (GUI).

In some aspects, the techniques described herein relate to one or more non-transitory computer-readable storage media including instructions that, when executed by one or more processors, cause the one or more processors to: receive, a first data object, the first data object including: an entity data set containing a plurality of entities; a first data set including request data associated with the plurality of entities; an event data set; and a plurality of data sets associated with one or more performance metrics; generate, based on at least one of the entity data set, the first data set, or the event data set, an entity data object for each of the plurality of entities; apply a machine-learning model to the entity data objects generated for the plurality of entities, the machine-learning model trained to identify a correlation between the entity data object for each of the plurality of entities and a probability of re-utilization of one or more resources; determine, based on the application of the machine-learning model to the entity data objects, a prediction indicator for each entity of the plurality of entities; assign, for each entity of the entity data object, an intervention flag based on the entity data object and the prediction indicator determined for the entity, the intervention flag including a management pathway; generate, a re-utilization offset data object for each of the plurality of entities, the re-utilization offset data object based on the prediction indicator determined for the entity, wherein the re-utilization offset data object includes information related to a total resource utilization associated with an intervention flag; and cause one or more of the re-utilization offset data objects generated for the plurality of entities to be displayed on a Graphical User Interface (GUI).

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1A is a diagram showing an example of a system configured for healthcare management, according to some embodiments of the disclosure.

FIG. 1B is a diagram of example components of a value impact platform, according to some embodiments of the disclosure.

FIG. 1C is a diagram of example components of a healthcare management module, according to some embodiments of the disclosure.

FIG. 2 is a flowchart showing a method for a flowchart showing a method for preventing or minimizing resource re-utilization, according to some embodiments of the disclosure.

FIG. 3 shows an example machine-learning training flow chart, according to some embodiments of the disclosure.

FIG. 4 illustrates an implementation of a computer system that executes techniques presented herein, according to some embodiments of the disclosure.

### DETAILED DESCRIPTION

The present disclosure relates to the field of data analytics and artificial intelligence. Various embodiments of this disclosure relate generally to techniques for predicting unnecessary and/or avoidable resource re-utilization, and, more particularly, to systems and methods for modeling predicted unnecessary resource re-utilization and interventions to increase efficiency of resource utilization.

As previously discussed, inefficient or unnecessary resource re-utilization is not only costly for organizations but often indicates suboptimal management of or offsite support for entities associated with the organizations

To address these concerns, a centralized system and method are provided which facilitate the comprehensive monitoring, analysis, and optimization of resource utilization within organizations. This centralized system harnesses a multitude of data sets, intertwining various attributes, events, and performance metrics of the entities linked with the organizations. By employing advanced analytical methodologies, such as machine-learning algorithms, the system is adept at identifying patterns and correlations that suggest inefficient resource allocation or utilization. Furthermore, these analyses not only provide insights but also actionable recommendations to improve the efficiency of resource distribution and utilization. Moreover, the systems and methods described herein leverage data that is unique to individual entities and addresses potential entity interventions at the entity-level. The system and method further include monitoring of the entity data and its changes over time, adjusting, updating, and retraining the applied models to account for changes in entity data, resulting in higher adoption of interventions, improved care pathways for the entities, and reduced unnecessary resource re-utilization. The above technical improvements, and additional technical improvements, will be described in detail throughout the present disclosure. Also, it should be apparent to a person of ordinary skill in the art that the technical improvements of the embodiments provided by the present disclosure are not limited to those explicitly discussed herein, and that additional technical improvements exist.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

Various non-limiting embodiments of the present disclosure will now be described to provide an overall understanding of the principles of the structure, function, and use of systems and methods disclosed herein for healthcare management outcomes.

Reference to any particular activity is provided in this disclosure only for convenience and not intended to limit the disclosure. A person of ordinary skill in the art would recognize that the concepts underlying the disclosed devices and methods may be utilized in any suitable activity. For example, while the present disclosure is in the context of healthcare management, one of ordinary skill would understand the applicability of the described systems and methods to similar tasks in a variety of contexts or environments. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Training the machine-learning model may include one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. After training the machine-learning mode, the machine-learning model may be deployed in a computer application for use on new input data that it has not been trained on previously.

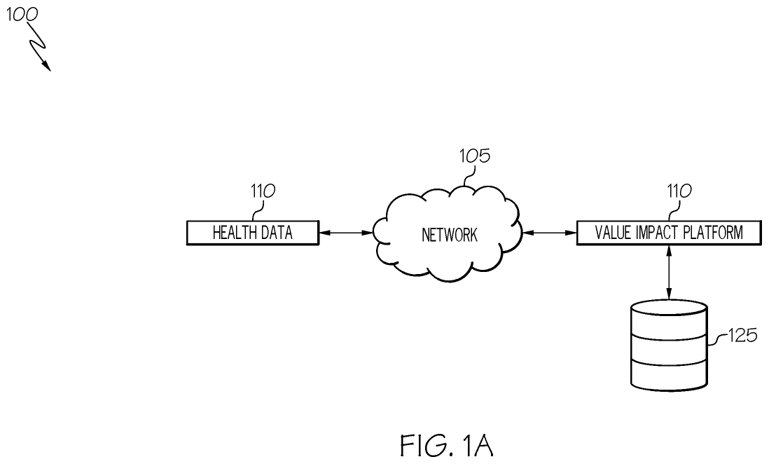

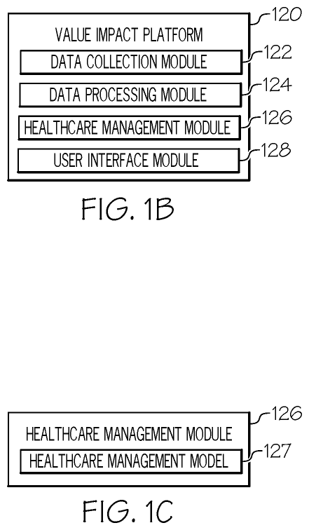

FIG. 1A is a diagram showing an example of a system that is capable of healthcare management, according to some embodiments of the disclosure. The depicted network environment, designated as 100, is in accordance with a specific embodiment of the current disclosure. The network environment 100 encompasses a communication infrastructure, such as network 105, which is accompanied by health data 110, and is further equipped with a value impact platform 120 integrated with a database 125.

In one embodiment, various components of the network environment 100 interact with each other through the network 105. The network 105 facilitates communication between the value impact platform 120 and one or more other systems, including one or more data sets, such as (but not limited to) health data 110. The one or more data sets and/or health data 110 includes data, one or more data entries and/or data objects associated with or comprising medical records. The network 105 includes one or more networks such as a data network, a wireless network, a telephony network, or any combination thereof.

The health data 110 encompasses an array of structured and unstructured information pertaining to the health of individuals. The health data, in some embodiments, is in the form of one or more data object, and encompass various facets, including but not limited to, health plan-provider contracts, member files, provider records, PCP to member attribution, medical and pharmacy claims, as well as insights from Impact Analytics, geographical and context based pricing indexes, Social Determinants of Health (SDoH), NYU Avoidable Preventable classification, Admit, Discharge, Transfers (ADT), Area Deprivation Index (ADI), Rural Urban (RUCA), risk and quality analytics, and the like. This diverse health data repository, comprising details such as demographic data, medical histories, insurance claims, and other health metrics, finds its repository in storage, which may take the form of local or remote data storage solutions, including file servers and cloud-based storage systems, among others.

The database 125 is used to support the storage and retrieval of data related to one or more data sets and/or data objects, such as the health data 110, storing metadata and/or healthcare data about one or more population represented in the health data 110, as well as any extracted information from the value impact platform 120. The database 125 can consist of one or more systems, such as a relational database management system (RDBMS), a NoSQL database, or a graph database, depending on the requirements and use cases of the network environment 100.

In one embodiment, the database 125 is any type of database, such as relational, hierarchical, object-oriented, etc., wherein data is organized in tables, lookup tables, or other suitable manners. The database 125 stores and provides access to data utilized by the value impact platform 120. The database 125 stores information related to the health data 110 as well as information generated by the value impact platform 120. The database 125 can store various types of information to aid in the healthcare management.

In one embodiment, the database 125 includes a machine learning-based training database that maps relationships, associations, connections, or the like between input parameters from the health data 110 and output parameters representing the one or more metrics for management of healthcare. For example, the training database can include machine learning algorithms that learn mappings between medical data inputs and one or more of resource utilization, resource re-utilization, adherence, sensitive condition treatment outputs, or the like. The training database can be routinely updated based on additional machine learning.

The value impact platform 120 communicates with other components of the network 105 using known or developing protocols. These protocols govern interactions between network nodes and define rules for generating, receiving, and interpreting information sent over communication links. The protocols operate at different layers, from generating physical signals to identifying software applications sending or receiving the information.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers.

In operation, the network environment 100 provides a framework for analyzing large amounts of health data 110, leveraging data analytics, artificial intelligence, and database technologies to support various use cases and applications. For example, the network environment 100 can be used to generate metrics, data objects, and insights from one or more data sets, such as the health data 110, based on user-defined criteria or a plurality of parameters.

To perform these tasks, the value impact platform 120 utilizes techniques such as the healthcare management model 127 (FIG. 1C), which analyzes the health data 110 and identifies one or more healthcare management metrics, which in some embodiments match one or more specified criteria. The value impact platform 120 can also utilize the data collection module 122 and data processing module 124 (FIG. 1B) to gather and prepare the health data 110.

To support storage and retrieval of data related to the healthcare management metrics, the database 125 stores metadata about the health data 110, such as data sources, types, and formats. The database 125 also stores information about the health management metrics output by the value impact platform 120, such as health criteria, identifiers, and statistics.

In addition to healthcare management, the network environment 100 can support other applications like data visualization, search, and predictive modeling. For example, the network environment 100 could allow users using user devices to search the health data 110 for one or more metrics matching certain criteria, or visualize healthcare metric statistics through interactive graphs and charts.

FIG. 1B is a diagram of example components of a value impact platform 120, according to some embodiments of the disclosure. Referring to FIG. 1B, the value impact platform 120 is a component of the network environment 100. The value impact platform 120 provides the capabilities to analyze one or more data sets, such as health data 110 and generate one or more data object including one or more healthcare management metric. As used herein, terms like “component” or “module” encompass hardware and/or software implemented by a processor or the like. For example, the value impact platform 120 includes components for collecting, processing, and analyzing health data as well as generating one or more data object including one or more healthcare management metrics. To that end, the value impact platform 120 includes modules such as a data collection module 122, a data processing module 124, a healthcare management module 126, and a user interface module 128. It is contemplated that the functions of these modules could be combined into fewer modules or performed by other modules with equivalent functionality.

In some embodiments, the data collection module 122 of the value impact platform 120 undertakes the collection of data from one or more data sets, such as health data 110, during the operation of the environment 100. The data collection module 122 is equipped to receive a myriad of data types such as, but not limited to, health plan provider contract data, provider data, member data including member eligibility data, PCP-to-member attribution data, medical and pharmacy claims data, proprietary or generated data, such as impact analytics data, pricing data, risk and quality analytics data, or the like, Healthcare Effectiveness Data and Information Set (HEDIS) quality metrics data, clinical prediction analytics, financial savings factors, Social Determinants of Health (SDoH) data, NYU Avoidable Preventable classification data, Area Deprivation Index (ADI) data, Admit, Discharge, and Transfer (ADT) data, Rural-urban Commuting Area (RUCA) data, proprietary episode treatment groupers (ETGs) data, proprietary service categories data, AHRQ groupers data, member geographic data, Drug Class Codes (DCCs), and the like.

In some embodiments, the health plan provider contract data includes, but is not limited to, the identification and credentials of providers, specifics of the health plans offered, a compilation of service and billing codes, agreed reimbursement rates, payment terms, the scope of benefit coverage, eligibility prerequisites for patients, protocols for authorizations and referrals, quality and performance benchmarks, procedures for dispute resolution, duration and termination information, privacy and confidentiality terms, regulatory adherence protocols, amendment procedures for the contract, utilization review guidelines, potential risk-sharing agreements, credentialing processes for healthcare providers, specifications regarding pharmacy formularies, and the like.

In some embodiments, the provider data includes, but is not limited to, identifiers such as names, addresses, contact details, specialties, qualifications, and tax identification numbers associated with providers. The data set also includes credentialing information, which verifies the qualifications and backgrounds of the providers, their affiliations with hospitals or other medical institutions, the insurance plans they accept, and their availability for patient appointments. In addition, the provider data contains historical data on the types and volumes of procedures performed, quality of care metrics, patient outcomes, and satisfaction scores, as well as data on billing practices and reimbursement rates.

In some embodiments, the member data includes, but is not limited to, identifiers such as names, birth dates, and member identification numbers associated with members. The member data further contains demographic details like addresses, contact information, gender, and employment information if relevant to the health plan. The health-related aspects of the data set cover a member's entire medical history with the plan, including plan enrollment dates, coverage details, dependents, benefit utilization records, and claims history. Additionally, the member data includes members' health conditions, diagnoses, treatment histories, and outcomes.

In some embodiments, the PCP-to-member attribution data includes, but is not limited to, one or more mappings between primary care providers (PCPs) and their attributed members, thereby identifying and/or linking individuals enrolled in a health plan and their designated primary caregivers. The PCP-to-member attribution includes data related to member identification numbers, names, and demographic information, alongside corresponding identifiers and credentials of the attributed PCPs. The PCP-to-member attribution data includes the duration of the member-PCP relationship, visit histories, and the nature of primary care services rendered. Additionally, in some embodiments, the PCP-to-member attribution includes data on care continuity, referral patterns, and the effectiveness of the PCP in managing the member's health, including preventative care and chronic disease management.

In some embodiments, medical and pharmacy claims data includes, but is not limited to, comprehensive records of members' interactions with healthcare systems, reflecting services rendered and pharmaceuticals provided. This includes data on claims submissions, detailing dates of service, types of services, service providers, claim amounts, and payment outcomes. Each entry correlates with member identification numbers and the associated healthcare providers or pharmacies. The medical and pharmacy claims also includes diagnostic codes, procedure codes, and pharmacy billing information, providing insights into the medical conditions treated and the medications dispensed. Furthermore, in some embodiments, the medical and pharmacy claims data includes a historical overview of members' claims over time, which can be analyzed to ascertain patterns in healthcare utilization, medication adherence, and the overall efficiency of healthcare services delivered.

In some embodiments, impact analytics data, such as data generated from a proprietary analytics engine, includes but is not limited to, one or more data sets which provide insights and/or data related to healthcare efficiency, costs, and outcomes. The impact analytics data aggregates and analyzes various aspects of healthcare services, encompassing medical claims, pharmacy claims, clinical data, and program participation records. The impact analytics data includes metrics on healthcare utilization, financial performance, clinical outcomes, and patient adherence to treatment regimens. Additionally, in some embodiments, this impact analytics data encompasses predictive analytics on risk stratification, care gaps, and potential interventions. The data set also integrates benchmarking against normative data or best practices, thereby enabling healthcare providers and payers to measure the effectiveness of their services against established standards.

In some embodiments, pricing data, such as data generated from a proprietary pricing engine, includes, but is not limited to, extensive data sets focused on the financial aspects of healthcare services. The pricing data encapsulates information on current market rates for various medical procedures and services, pharmaceutical pricing, and the costs associated with different healthcare providers. The pricing data comprises details on negotiated contract rates, reimbursement models, historical pricing trends, and comparative analysis across different regions or service providers. Additionally, in some embodiments, the pricing data may integrate cost forecasting, budget impact models, and scenario analyses.

In some embodiments, risk and quality analytics data, such as data generated from a proprietary analytics engine, includes but is not limited to, an array of data points that enable evaluation and monitoring of the quality, efficiency, and safety of healthcare delivery that encompasses risk assessments, quality measures, patient safety indicators, and compliance with clinical guidelines. The risk and quality analytics data includes outcomes data, risk adjustment factors, and analytics related to population health management. Additionally, in some embodiments, the risk and quality analytics data includes data related to care management programs, member health assessments, and provider performance evaluations.

In some embodiments, Healthcare Effectiveness Data and Information Set (HEDIS) quality metrics data includes, but is not limited to, one or more standardized performance measures that are used to assess the quality of care and services provided by health plans. The HEDIS quality metrics data includes one or more indicators across various domains of care, including preventive health services, chronic disease management, mental health care, substance use treatment, care coordination, and the like. The HEDIS quality metrics data includes data related to healthcare effectiveness, patient safety, timeliness of care, and patient engagement. Additionally, in some embodiments, the HEDIS quality metric data may also encompass measures of utilization and risk-adjusted health outcomes.

In some embodiments, clinical prediction analytics data includes, but is not limited to, patient demographics, historical clinical data, treatment records, real-time health monitoring data, and the like. The prediction analytics data, in some embodiments, includes data generated by one or more predictive models and algorithms that analyze patterns in the data to anticipate future health events, such as hospital readmissions, disease progression, or the likelihood of specific health conditions developing. Additionally, in some embodiments, the clinical prediction analytics data includes data indicative of risk scores, potential gaps in care, and suggested preventative measures.

In some embodiments, financial savings factors data includes, but is not limited to, data related to cost avoidance, reduction in unnecessary medical procedures, efficiencies gained through improved care coordination, and savings from formulary management in pharmacy benefits. Additionally, in some embodiments, the financial savings factors data includes data on member cost-sharing amounts, provider network contracting savings, and the impact of wellness programs on overall healthcare costs.

In some embodiments, Social Determinants of Health (SDoH) data includes, but is not limited to, data points related to non-medical factors influencing patient health outcomes. The SDoH data encompasses socio-economic status, education level, neighborhood and physical environment, employment status, social support networks, and the like. The SDoH data, in some embodiments, includes information collected through patient surveys, community health assessments, and public health databases. Additionally, in some embodiments, the SDoH data includes indicators of health disparities, access to healthcare services, and environmental risk factors.

In some embodiments, NYU Avoidable Preventable classification data includes, but is not limited to, data related to metrics that categorize healthcare events deemed either avoidable or preventable with proper and timely medical care, patient education, and other interventions. This classification data includes data elements such as emergency department visits that could be managed in primary care settings, hospital admissions for conditions preventable through outpatient services, and incidences of chronic disease complications that can be mitigated through proper management and lifestyle adjustments.

In some embodiments, Area Deprivation Index (ADI) data includes, but is not limited to, data that ranks neighborhoods by socioeconomic status disadvantage in a region or across the nation. The ADI data includes data related to income, education, employment, housing quality, and other socioeconomic factors which demonstrate disparities across different regions. The ADI data, in some embodiments, is arranged by region, such as by zip code.

In some embodiments, Admit, Discharge, and Transfer (ADT) data includes, but is not limited to, operational data detailing patient movement within a healthcare facility or across facilities. This data set includes timestamps and related information for patient admissions, discharges, and transfers among different departments or care settings. The ADT data, in some embodiments, is collected in real-time, facilitating immediate updates to a patient's status and location. Additionally, in some embodiments, the ADT data includes identifiers that can be used to track patient flow, manage bed occupancy, and coordinate care transitions effectively.

In some embodiments, Rural-urban Commuting Area (RUCA) data includes, but is not limited to, data that categorizes regions, such as U.S. census tracts, using measures of population density, urbanization, and daily commuting. The RUCA data, in some embodiments, provides data relating to the rural-urban continuum, distinguishing between areas, such as metro and rural. Additionally, in some embodiments, the RUCA data includes the primary commuting flows to identify the social and economic integration of locales

In some embodiments, Drug Class Codes (DCC) data includes numerical and alphabetical identifiers that categorize drugs based on their pharmacological properties, therapeutic effects, chemical structure, and mechanism of action. The DCC data is structured to represent relationships between different drugs and their respective classes, enabling the identification of similar or related compounds. The DCC data is stored in a database where each drug is linked to one or more drug class codes, which in turn are associated with detailed descriptions of the drug class characteristics.

The data is ingested into the system via multiple pathways, thereby providing flexibility in the collection mechanism. Specifically, one pathway includes an Application Programming Interface (API) that establishes a secure communication channel for automated data transfer between the data collection module 122 and external data sources, thus facilitating real-time or batch-based data acquisition. Another pathway allows for manual input by authorized users via a dedicated user interface, where such input can be executed through file uploads or direct data entry into predefined fields. Additionally, data intake can be accomplished through third-party integrations, middleware, or direct database queries that serve to populate the database 125. The data collection module 122 further incorporates data validation and integrity checks to ensure the consistency and reliability of the ingested data. By offering a plurality of data intake methodologies, the data collection module 122 ensures robust and comprehensive data assimilation for downstream processing.

The data processing module 124 of the value impact platform 120 partakes in the processing and preparation of the data for further analysis by the healthcare management module 126. The data processing module 124 engages in the cleaning of the data, removal of irrelevant or redundant information, and conversion of the data into a format suitable for further processing by the healthcare management module 126. The data processing module 124 is configured to augment the initial data collection by transforming the raw, heterogeneous data into a unified, standard format, which is useful for accurate and efficient downstream processing. Specifically, the data processing module 124 executes a series of algorithms responsible for data standardization, thereby reconciling discrepancies in data types, units, or terminologies originating from disparate sources.

The data processing module 124 also integrates error-handling mechanisms to identify and rectify potential data inaccuracies or anomalies. Such mechanisms may involve rule-based checks, probabilistic data matching, or data imputation techniques, all aimed at preserving data quality and integrity. Furthermore, the data processing module 124 may incorporate parallel processing capabilities to concurrently handle multiple data streams, thereby ensuring timely and efficient data throughput. This is particularly advantageous when dealing with large-scale data sets or real-time analytics where swift data processing is desired.

The healthcare management module 126, upon receiving the prepared data from data processing module 124, applies algorithms and models, such as healthcare management model 127, to generate one or more data object including one or more healthcare management metric, based on the input data. The healthcare management module 126 utilizes various algorithms and employs a variety of models to accomplish its task. The healthcare management module 126 engages in the computational manipulation of the ingested data. Utilizing the healthcare management model 127 as one among a possible array of analytical frameworks, the healthcare management module 126 applies a combination of algorithmic and machine-learning methodologies to generate one or more healthcare management metrics based on the input data. Such metrics serve as quantifiable representations of various aspects of healthcare management.

In one embodiment, the healthcare management module 126 applies algorithms related to clinical opportunities methodology. This methodology integrates diverse sets of processed data, such as medical claims, financial data, and clinical histories, to produce a healthcare management metric that reflects opportunities for cost and quality optimization in healthcare delivery.

In another embodiment, the healthcare management module 126 employs machine-learning-based prediction algorithms to produce metrics that predict future healthcare events. These could include patient risk stratification or likelihood of hospital readmission, or the like. The predictive models, which are a part of the healthcare management model 127, use features extracted from the processed data, such as social determinants of health, historical medical data, area deprivation index scores, one or more other features extracted from the processed data as discussed herein, or a combination thereof.

Additionally, the healthcare management module 126 in some embodiments uses value impact modeling to generate healthcare management metrics that evaluate the resource efficiency (such as economic, staffing, or material usage implications) of distinct clinical interventions or pathways. These metrics are derived from simulations that are conducted using various models, each designed to measure the financial impact of specific healthcare decisions.

The healthcare management module 126, in some embodiments, further produces healthcare management metrics that represent aggregated patient worklists or next-best-action recommendations. These metrics are formulated through a combination of rule-based algorithms and probabilistic models, which evaluate and incorporate variables like HEDIS quality metrics and medical and pharmacy claims.

After the healthcare management module 126 has generated the one or more data objects including one or more healthcare management metric based on the input data, a user interface generated on a user device via the user interface module 128 displays the results to the user at an appropriate time. The user interface provides an interactive and intuitive interface, enabling the user to view, modify, or confirm the generated results. The user interface also enables the user to provide feedback or additional information to improve the healthcare management process or adjust the healthcare management model 127 accordingly. The user interface module 128 is also configured to receive a user input via an interactive interface, the user input being one or more parameters.

FIG. 1C is a diagram of example components of a healthcare management module 126, according to some embodiments of the disclosure. FIG. 1C provides a more detailed view of the healthcare management module 126 and its relationship with the healthcare management model 127 within the value impact platform 120. As depicted, the healthcare management module 126 includes a healthcare management model 127. The healthcare management model 127 is configured or trained to determine appropriate healthcare management metrics, in the form of one or more data object, related to resource utilization, care adherence, care outcomes, resource efficiency, and the like, based on various factors, such as those reflected in the health data 110. Furthermore, the healthcare management model 127 also takes into account changes to the health data and/or to the populations within the health data to increase the likelihood of an accurate response.

The healthcare management model 127, as part of the healthcare management module 126, orchestrates the creation of healthcare management metrics, such as data objects, from health data 110. This algorithm is agnostic to its underlying implementations and is designed to accommodate various types of algorithms, either individually or in combination, to achieve the desired outcomes. In some embodiments, the healthcare management metrics generated by the healthcare management model 127 pertain to predicted utilization of resources and services, projected complexity of medication regimens, identified categories associated with risks and/or severities, or other relevant aspects related to patient care and treatment planning. It should be noted that while the described implementation involves a predicative model, alternative configurations incorporate other models or approaches depending upon the specific needs and requirements of the healthcare facility and patients served. For example, the healthcare management model 127, in some embodiments, analyzes historical patterns in healthcare usage data to develop predictions about future trends. This information is then be used to optimize staffing levels, inventory management, equipment maintenance schedules, and other logistical considerations necessary for providing efficient and effective medical care. Additionally, the generated metrics assist clinicians in identifying patients who would benefit from targeted interventions or early discharge planning efforts, thereby reducing hospital stays and improving overall patient health outcomes.

In some embodiments, the value impact platform 120 is configured to support contract ingestion and standardization. The data collection module 122 is configured to receive contract terms among other types of healthcare-related data. Upon collection, these contract terms are forwarded to the data processing module 124. The data processing module transforms the heterogeneous contract data into a unified, structured format that is suitable for subsequent processing by the healthcare management module 126 and storage within the database 125.

The data processing module 124 employs algorithms designed specifically for contract standardization. These algorithms reconcile variances in contract terminologies, units, and conditions, thereby eliminating inconsistencies that could potentially impact the quality of the generated healthcare management metrics. This standardization process results in the normalization or standardization of contracts from disparate sources that can be accurately compared, analyzed, and integrated within the healthcare management framework enabled by the value impact platform 120.

In addition to terminology reconciliation, the data processing module 124 performs the task of structuring the ingested contract data. This involves the breaking down of complex contract clauses into constituent elements, which are then mapped to predefined fields within the database 125. By doing so, the data processing module 124 ensures that the contract data is organized in a manner conducive to efficient query execution and data retrieval. Following the completion of the contract ingestion and standardization process, the standardized contract data is stored in the database 125 and is made accessible to the healthcare management module 126 for subsequent analytical operations.

In some embodiments, the data processing module 124 is configured to combine two or more contracts for the purpose of generating healthcare management metrics. The platform identifies contracts with terms sufficiently similar to warrant amalgamation into a single data object. Subsequently, these unified contract data objects are stored in the database 125 and are rendered accessible to the healthcare management module 126 for further analytical activities. The data processing module 124 incorporates rules-based mechanisms or utilizes one or more models or algorithms to establish the suitability of combining specific contracts. In a rules-based approach, pre-defined combination rules are set by one or more users of the system. These rules specify criteria that contract terms must meet to be considered similar, such as identical service categories, payment models, geographical locations, or the like. In some embodiments, the data processing module 124 employs computational models or algorithms to assess the suitability of contracts for combination. These algorithms analyze attributes such as contract duration, parties involved, and other contractual elements, and apply statistical or machine-learning techniques to make determinations on whether contracts can be combined together.

Once contracts are combined into single data objects, the user, through the user interface module 128, is enabled to select these combinations for analysis. The healthcare management module 126 then generates one or more healthcare management metrics or reports based on the combined contract data objects. The system is further designed to allow the user to modify the selection of combined contracts. Upon such re-selection, the healthcare management module 126 automatically re-generates the healthcare management metrics or reports to reflect the updated corpus of selected contracts.

In some embodiments, the value impact platform 120 generates one or more performance reports for the individual or combined contracts. This performance reporting is formulated based on a combination of input data and the standardized or amalgamated contract data objects stored in the database 125.

In one embodiment, the healthcare management module 126 employs algorithms related to financial performance reports. These algorithms integrate the standardized contract data with other forms of healthcare data, such as medical and pharmacy claims, HEDIS quality metrics, clinical prediction analytics, or the like, to yield a performance report that assesses the financial implications of the individual or combined contracts. The report covers aspects such as cost-efficiency, quality of care, and adherence to contract terms, among other criteria.

In another embodiment, the healthcare management module 126 uses the clinical opportunity identification methodology to generate performance reports. This methodology combines the contract data, whether individual or combined, with clinical histories, social determinants of health, or other relevant healthcare data to identify opportunities for clinical improvements and cost savings. The resulting performance report would provide a granular analysis of the efficacy and efficiency of healthcare service delivery as stipulated by the contract terms.

For contracts that have been combined, the healthcare management module 126 is configured to generate a unified performance report that represents the aggregated impact of the bundled contracts. This unified report would comprise metrics such as cumulative cost savings, overall quality improvement, and combined compliance rates, synthesized from the individual contracts included in the combination.

Further, in some embodiments, the system enables the user, through the user interface module 128, to interact with the generated reports. Users can select different combinations of contracts, prompting the healthcare management module 126 to re-calculate and re-generate performance reports based on the newly selected combinations. This adaptability ensures that users obtain tailored insights that cater to different analytical needs.

In some embodiments, the healthcare management module 126 is configured to perform tasks related to clinical and quality modeling. The module receives input data and standardized or combined contract data from the database 125 and applies a series of algorithms and models for the generation of clinical and quality metrics. These metrics pertain to the assessment of healthcare services, patient outcomes, and compliance with established healthcare standards. The healthcare management module 126 incorporates specific algorithms designated for evaluating quality metrics such as HEDIS scores, patient satisfaction rates, and clinical effectiveness measures. These algorithms integrate with the contract data to discern how specific contractual terms and conditions influence quality outcomes. For example, an algorithm assesses how a payment model specified in a contract impacts the healthcare provider's adherence to HEDIS standards. Similarly, the module includes clinical modeling capabilities that employ advanced algorithms or machine-learning models. These clinical models incorporate multiple variables from the input data, including but not limited to medical and pharmacy claims, member eligibility, and social determinants of health, to produce actionable insights. For instance, the module could utilize an algorithm that integrates patient medical histories and contract-specific guidelines on pharmaceutical usage to determine optimal drug regimens for individual patients. Moreover, the clinical and quality metrics generated can be included as part of broader performance reports. These reports are displayed through the user interface module 128, which allows users to interact with and interpret the metrics, thereby enabling more informed healthcare management decisions.

In instances where combined contracts are used, the healthcare management module 126 is further configured to generate clinical and quality models that reflect the aggregate effect of these combined contracts. For instance, a unified quality model might be generated that blends the quality metrics from multiple contracts to offer a holistic view of healthcare service quality across an entire healthcare network.

In some embodiments, the healthcare management module 126 incorporates functionalities designed for dynamic scenario modeling. Specifically, the module enables the modeling of scenarios that simulate the impact of various improvement opportunities on performance metrics, particularly with respect to financial, clinical, and quality dimensions. This capability allows users to forecast the outcomes of potential actions or interventions within the healthcare system. For instance, the dynamic scenario modeling employs a modeler which is configured to capture the top n common payer scenarios. This modeler assimilates information from diverse data sources such as financial models, clinical histories, and quality metrics, all of which are stored in the database 125. The modeler then utilizes these data points in conjunction with the contract data, whether individual or combined, to generate a set of scenario options.

Understanding that medical groups often operate under resource constraints, the dynamic scenario Modeler allows the user to selectively focus efforts on one or two metrics with the objective of optimizing performance against contractual targets. Users interact with this feature via the user interface module 128, where they can specify the level of resource allocation they wish to devote to particular opportunities for improvement. For example, in some embodiments, a user might elect to focus on optimizing HEDIS quality metrics. The dynamic scenario Modeler would then simulate the impact of such an optimization on financial performance, considering parameters such as reimbursement rates stipulated in the contract or contracts. Simultaneously, the dynamic scenario modeler would also forecast the implications on clinical performance metrics, such as patient health outcomes or admission rates. By way of another example, in some embodiments, the user could decide to emphasize efforts on cost-saving measures in pharmaceutical spending. Here, the dynamic scenario modeler generates a scenario illustrating how such an effort would affect not just financial metrics like overall spending, but also quality metrics like patient satisfaction and clinical efficacy. In cases involving combined contracts, the dynamic scenario Modeler is further configured to aggregate the impacts across the multiple contracts, providing a consolidated view of how resource allocation in selected areas would influence performance metrics at a holistic level.

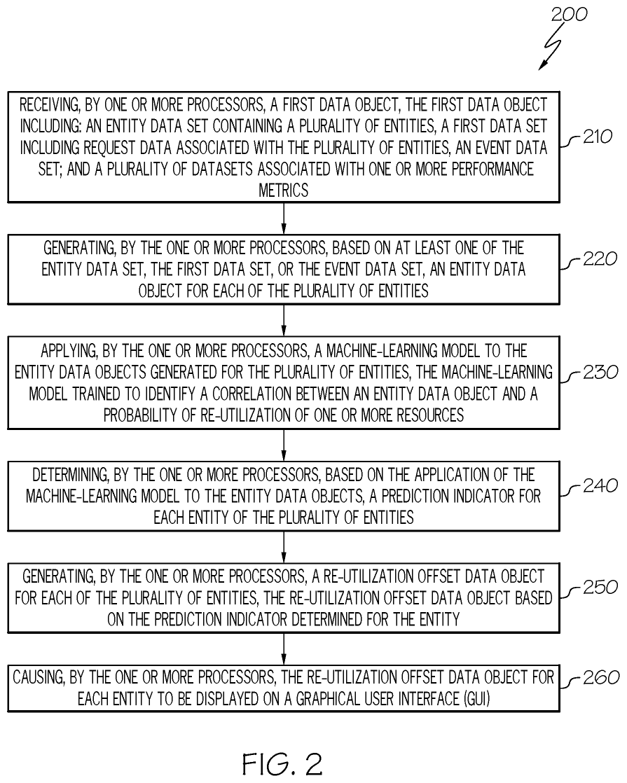

FIG. 2 is a flowchart showing a method 200 for preventing or minimizing resource re-utilization (e.g., health-related readmissions). In some embodiments, the value impact platform 120 receives a first data object. The first data object may comprise one data object or a plurality of data objects, that includes a collection of data sets. In some embodiments, the first data object includes an entity data set containing a plurality of entities. The entity data set encompasses data about one or more members, with each member potentially being associated with one or more providers, such as a healthcare provider. This first data object further incorporates a first data set comprising request data related to the plurality of entities. Notably, the request data, which pertains to medical claims filed by the members, carries information concerning historical claims for these members.

The first data object also comprises an event data set. This event data set is versatile, allowing for the inclusion of one or more data arrays, such as an episode treatment groupers (ETGs) array, a service categories array, or the like. In some embodiments, ETGs are a classification system used in healthcare to group clinically similar medical events. The ETGs array includes information which is utilized and applied to medical events, such as medical claims data, to aggregate individual, patient-specific medical claims and encounter data into clinically meaningful and discrete units, known as “episodes of care.” Each episode represents a distinct phase of a patient's medical treatment, from initial diagnosis through the course of treatment for a particular condition.

The service categories array is a systematic classification framework to categorize various medical services and procedures. This array breaks down the multitude of healthcare services into more manageable and distinct categories, making it easier to analyze and understand healthcare operations. Within the service categories array, individual services or procedures are grouped based on their nature, purpose, or the medical specialty they pertain to. For instance, services related to cardiology, neurology, orthopedics, or radiology might each form distinct categories. Moreover, the array could further classify services based on factors like the type of care (e.g., preventative, diagnostic, therapeutic), setting (e.g., inpatient, outpatient), or the severity and complexity of the case. By employing such a categorization system, healthcare providers, payers, and analysts can achieve a clearer perspective on the distribution, utilization, and costs associated with different types of medical services.

Furthermore, in some embodiments the event data set encompasses a data records array connected with admissions, discharges, and transfers (ADT). In some embodiments, the ADT integrates a primary diagnosis associated with one or more connected admissions, discharges, and transfers. In some embodiments, the ADT (Admission, Discharge, Transfer) array includes comprehensive details of a patient's movement within and/or between healthcare facilities. This might encompass data related to the time and reason for the patient's initial admission, the specific departments or wards they were admitted to, and any subsequent internal transfers between units or specialties. Additionally, the ADT array could capture data on the patient's medical condition or diagnosis upon admission, as well as any changes in diagnosis or additional conditions identified during their stay. Information about planned or unplanned discharges, including the reason for discharge (e.g., medical improvement, transfer to another facility, or end-of-life decisions) and post-discharge instructions or follow-up care recommendations, could also be integral components of the ADT array. Furthermore, the array in some embodiments stores details about the attending physicians, nursing care plans, medications administered during the stay, and any special equipment or interventions used.

The ETG array, in some embodiments, relates to the ADT array such that the ETG enables classification of the events within the ADT. For example, ETGs might be applied to ADT (Admission, Discharge, Transfer) data to group and categorize all related hospital activities during a patient's stay, providing a holistic view of the entire care episode from admission through discharge or transfer.

The first data object is also supplemented by a plurality of data sets associated with one or more performance metrics. These performance metrics include additional health data, consisting of a provider data set which relates to one or more providers, insurers, hospital service locations, or the like. In some embodiments, the plurality of data sets associated with one or more performance metrics includes a social determinants of health (SDoH) data set. In some embodiments, the SDoH (Social Determinants of Health) data set includes a broad spectrum of non-medical factors that influence health outcomes for one or more members within the entity data set. This encompasses socio-economic data such as income levels, educational attainment, employment status, and housing stability. It also includes data about a patient's social and community context, including social support networks, community engagement, and potential exposure to violence or crime. Environmental factors, such as access to clean water and safe housing, proximity to parks or recreational areas, and potential exposure to environmental toxins, are, in some embodiments, also included in the SDoH data set. The SDoH data set, in some embodiments, also contains indicators and/or data related to access to health care, including proximity to healthcare facilities, transportation options, and health insurance status. Behavioral data, including dietary habits, physical activity levels, tobacco and alcohol use, and other substance use or misuse, are, in some embodiments, present.

In some embodiments, an Area Deprivation Index (ADI) data set is included in the plurality of data sets associated with performance metrics. The ADI data set provides indicators pertaining to geographical regions reflecting socio-economic challenges based on factors such as income and education levels. Specifically, the ADI data set contains indicators that rank regions based on their deprivation scores. These scores are derived from comprehensive evaluations of various factors, including but not limited to, household income, employment rates, access to education, and other socio-economic determinants. Such data assists in recognizing regions where residents might be at a higher risk for health disparities due to socio-economic challenges. When integrated with other data, such as the entity data set or the event data set, the ADI data set offers a deeper context, potentially highlighting correlations between geographical deprivation and health outcomes. This, in turn, aids the value impact platform 120 in generating more holistic and accurate prediction indicators.

In some embodiments, a rural/urban data set (RUCA) comprising rural-urban commuting area codes is included in the plurality of data sets associated with performance metrics. The RUCA data set delineates geographical regions based on how urban or rural the geographical region is and the nature of work-related commutes. Specifically, these codes categorize regions into urban, rural, transitional areas, and the like, offering insights into the dynamics of population density, infrastructure, and accessibility to health and other services. The RUCA data set can be utilized to determine the relative availability and reach of healthcare facilities, transport systems, and local amenities within these designated areas.

Furthermore, when interfaced with the entity data set or the event data set, the RUCA data set serves to contextualize health data 110 within the broader framework of urban-rural divides. This aids in highlighting disparities in healthcare access, service quality, and health outcomes across different community types. For instance, members from rural areas may have different healthcare needs or face different challenges than those in urban locales, such as longer travel times to health facilities or limited access to specialist care.

In some embodiments, a Major Practice Categories (MPCs) data set is included in the plurality of data sets associated with performance metrics. In some embodiments, the Major Practice Categories (MPC) data set contains categorized clinical information that is derived from Episode Treatment Groups, encompassing details such as the nature of medical interventions, patient diagnoses, treatment outcomes, and related healthcare services pertinent to patient admissions and readmissions.

In some embodiments, at step 220, the value impact platform 120 generates, based at least in part on the entity data set, the first data set, or the event data set, an entity data object for each of the plurality of entities. In some embodiments, the generation of the entity data object integrates diverse sets of data to create a comprehensive record for each entity, which in the context of this method, typically represents a member. This generation process, as an example, consolidates entity data that includes specifics about a member, the member's historical medical claims, event-based data, and metrics that evaluate performance.

In certain embodiments, the entity data object is structured to collate member-related information, pulling from various data sources such as the entity data set which might contain data about associations between a member and healthcare providers. Concurrently, the first data set, which pertains to medical claims, supplies details concerning previous claims related to the member. This includes particulars of past treatments, medication claims, surgical procedures, or the like.

In relation to the event data set, specific arrays, like the episode treatment groupers (ETGs) array, service categories array, or the data records array related to admissions, discharges, and transfers (ADT), are integrated into the entity data object. This allows for a meticulous capture of data, for instance, correlating ETGs with each of the medical claims or applying service categories to individual medical claims. In some embodiments, this approach ensures that the most pertinent data, such as a primary diagnosis linked with ADT, is embedded within the entity data object.

When applying the episode treatment groupers (ETGs) to the medical claims, the aim is to categorize the claims into clinically meaningful treatment groupings. Each medical claim, which might represent a distinct medical procedure, service, or treatment that a member has undergone, is mapped to a specific ETG and the entity data object is updated to reflect the mapping and categorization. This categorization process considers variables like diagnosis codes, procedure codes, and the place of service associated with each claim. Similarly, in some embodiments, service categories are applied to the medical claims to map the medical claims to one or more service categories, at which point the entity data object is updated to reflect the service categorization.

Furthermore, depending on the sequence of events during a member's admission cycle, unique entity data objects can be generated. In certain embodiments, each major event like admission, transfer, or discharge triggers the creation of a new or updated entity data object. These objects not only act as repositories of data but also facilitate easy interfacing with other computational models, such as machine-learning models, which use the entity data object, or at least a portion thereof, as an input for predictive analytics or the like.

The resultant entity data object is one or more vectors and/or arrays, representing a consolidated and structured representation of diverse data points associated with an entity, typically a member (such as a patient). By designing it as such, the data object can be ingested by computational models, ensuring that each dimension of the vector or array corresponds to a specific feature or data point pertinent to the member's health profile, historical data, and events. The structure and composition of this entity data object are particularly conducive for machine learning algorithms, where each dimension of the vector or array corresponds to an input node of the machine learning model.

At step 230, the entity data objects, which encapsulate the structured and consolidated data regarding the members' healthcare, are applied to (or fed to) a machine-learning model. The design and architecture of the model are trained to decipher patterns, relationships, and insights from the multi-dimensional data present in the entity data objects. Depending on the specific objectives and outcomes desired from the method, this machine-learning model is in some embodiments a supervised model, trained using labeled data to predict specific outcomes, or an unsupervised model that identifies patterns and clusters within the data without pre-labeled examples. In one embodiment, a Random Forest-based classification model is used as the machine-learning model.

When the entity data objects are applied to the model, the algorithms ingest the information, weighing different features based on their relevance and potential correlation with desired outcomes. For instance, if the goal is to predict the likelihood of readmission for a member, the model would prioritize features in the entity data object that historically have shown strong correlations with readmission patterns. Over time, with the continuous feeding of more entity data objects, the machine-learning model undergoes iterative refinement, enhancing its prediction accuracy and ensuring that it stays updated with evolving healthcare data trends.

Furthermore, the flexibility of machine learning allows for the incorporation of different types of models or hybrid models, depending on the complexity of the data and the desired outcome. For example, a neural network might be employed to capture intricate relationships between features, or a decision tree might be used for its interpretability in scenarios where understanding the decision-making pathway is preferred. Regardless of the model type, step 230 ensures that insights derived are both robust and actionable, fostering data-driven decision-making processes in healthcare management or the like.

The machine learning model, in some embodiments, is a single model, while in some embodiments, the system selects from one or more machine learning model alternatives based on when the entity data object was generated. For example, some embodiments of the machine learning model are calibrated specifically to decipher patterns from entity data objects generated during a patient's admission. In some embodiments, the machine learning model is trained to analyze entity data objects generated at the point of discharge. In some embodiments, the machine learning model is trained to analyze entity data objects at the time of transfer.

In some embodiments, the machine learning model is a single model that includes a mixture-of-experts framework. In such embodiments, the model consists of multiple specialized nodes or vectors, or clusters of nodes or vectors, each tailored for a specific ADT event. These components collaborate, allowing for a nuanced analysis that leverages the strengths of each specialized node.

In some embodiments, the utilization of one or more resources is a readmission of the member. The utilization is, in some embodiments, expressed as a probability output that the member is readmitted over a pre-determined future timeframe. In some embodiments, that time frame is 30 days, but it will be appreciated that the time frame can be adjusted depending on the needs of the user.

At step 240 in FIG. 2, the value impact platform 120 determines, based on the application of the machine-learning model to the entity data objects, a prediction indicator for each entity of the plurality of entities. This prediction indicator serves as a quantifiable metric that is representative of the anticipated behavior or outcome for a specific entity, such as the likelihood of a health-related readmission within a specific time period. Typically, the pre-determined time period for gauging the likelihood is set to 30 days post-discharge, although it should be understood that other time periods could also be contemplated depending on specific applications or scenarios.

In some embodiments, once the machine-learning model has been applied to the entity data object, which pertains to a specific member, the generated prediction indicator is then added or incorporated into that same entity data object. This integration facilitates streamlined data management and retrieval, ensuring that for each entity, all relevant data and predictions are consolidated in a singular entity data object.

The nature of the prediction indicator, in some embodiments, is a numeric score. This score is computed such that it directly corresponds to the likelihood that the respective entity, or member, will engage in the re-utilization of a resource during the pre-determined time period. This numeric score, which in some embodiments ranges from 0 to 1 or is on a scale of 0 to 100, for instance, or is expressed in a percentage, offers healthcare professionals a clear and immediate understanding of the risk profile associated with each member. High scores might indicate a higher likelihood of resource re-utilization. Conversely, lower scores could suggest that the member is on a favorable health trajectory and might not require as intensive monitoring or intervention.

In some embodiments, the value impact platform 120 then generates a list of patients which have any risk for all-cause readmissions for a pre-determined future time period, such as 30 days. The value impact platform 120 then compares the list of patients against a period ADT data set, such as a daily admission, discharge, transfer data file to identify patients who both are at risk of readmission and appear on the periodic ADT data set. These patients are assigned a flag, which is utilized to identify these patients, such as to assign a higher priority to these patients, sort these patients in a list or user interface element, or the like.

At step 250 of FIG. 2, the value impact platform 120 generates a re-utilization offset data object for each entity from the plurality of entities. Therefore, in some embodiments, a plurality of re-utilization offset data objects are generated for the plurality of respective entities. This re-utilization offset data object integrates a re-utilization offset metric. This metric is based on, at least in part, by the prediction indicator determined for the entity as explained in step 240.

In some embodiments, the re-utilization offset data object provides a quantified measure that represents potential monetary savings if a healthcare intervention is executed for the entity. This potential savings quantification integrates data regarding alternative care pathways. It further compares the costs associated with these pathways against potential readmission costs, thereby offering a comparative financial perspective.

The derivation of the potential cost savings within the re-utilization offset data object, in some embodiments, further considers the probability that the selected alternative care pathway will obviate the need for a subsequent readmission. This probability determination in some embodiments is based at least in part on the prediction indicator and assimilates data regarding the entity's historical medical interactions and present health conditions, along with one or more additional healthcare metrics as discussed herein. In some embodiments, as interventions are put into practice on the members, the actual success rates of the interventions are applied back to the system to update the probabilities and provide a constantly updating model of the probability that the selected alternative care pathway will obviate the need for a subsequent readmission.

In some embodiments, the value impact platform 120, in generating one or more re-utilization offset data objects, performs a savings opportunity modeling process. In some embodiments, the savings opportunity modeling process involves utilizing a Major Practice Categories (MPCs) data set, which are based on one or more ETG data sets for classifying clinical aspects of both initial admissions and subsequent readmissions. This classification provides a clinical context for each admission event. The process also employs a first algorithm, such as an Identified Index (II) readmissions algorithm, to analyze historical readmission data. In some embodiments, the value impact platform 120 utilizes the II readmissions algorithm in analyzing patient admission records to identify instances of readmission. The value impact platform 120 assesses patient data, including previous admission dates, diagnoses, treatments, and discharge information. The II algorithm cross-references this data with subsequent admissions to determine if they qualify as readmissions based on predefined criteria, such as the time elapsed between admissions and the similarity of diagnoses. The II algorithm is designed to discern patterns in readmissions. The value impact platform 120 further compares identified readmission costs associated with the historical readmission data against an offset analysis proprietary tool, which includes offset costs derived from clinical judgments informed by literature reviews. The value impact platform 120 applies these offsets to all readmissions, with the clinical literature providing a basis for determining the percentage of readmissions that are potentially reducible through the application of MPC-based interventions. This modeling by the value impact platform 120, therefore, combines clinical classifications, historical data analysis, and literature-informed judgments to assess and apply cost offsets in the context of readmissions. As a result, the value impact platform 120 generates one or more re-utilization offset data objects which include one or more offset costs associated with data generated during the savings opportunity modeling process.

At step 260, the value impact platform 120 causes one or more of the re-utilization offset data objects generated for the plurality of entities to be displayed on a Graphical User Interface (GUI). The interface incorporates various display elements, such as charts, tables, graphs, or numerical indicators, providing the end-user with an intuitive understanding of the data. This data can encompass the potential cost savings, predicted re-admission risks, and the recommended alternative care pathways. These elements are constructed to be interactive, allowing users to delve deeper into specific data points, modify viewing parameters, or extract granular details, all of which enhance decision-making processes.

In some embodiments, a cluster data object is generated. The cluster data object utilizes one or more clustering algorithms to group the entity data objects based on common features. The resultant cluster data object include members that are unique to each cluster data object, allowing for interventions and care pathways to be broadly applied to multiple members by applying them to the cluster data object as a whole.

In some embodiments, one or more intervention data objects and/or arrays are generated. The intervention data object and/or array, in some embodiments, includes one or more cluster data objects and the members associated with the one or more cluster data objects. For each member, the processor assigns one or more interventions to the member. The intervention, in some embodiments, is associated with one or more alternative paths of care, which is associated with a particular resource utilization or avoidance of certain instances of resource re-utilization. The intervention is assigned utilizing one or more machine-learning models and/or algorithms to output an intervention that results in the most efficient resource utilization, such as by suggesting an intervention by diverting the member to an alternative care pathway based on one or more member data, the likelihood of success of the intervention, the expected resource utilization (such as cost) of the intervention, and the overall reduction in resource utilization of the alternative care pathway.

In some embodiments, the generation of the intervention by the processor is based on the probability of readmission for the member, such as the generated percentage previously described herein. The percentage indicates a probability of readmission over a future time frame, such as the next 30 days. In some embodiments, the intervention data object is generated by the processor by applying a machine-learning model to the generated probability of readmission (e.g., the prediction indicator) and one or more of the member data, the generated entity data objects, the flagged patients list, or one or more other data generated or received by value impact platform 120. In some embodiments, the intervention list is applied by a user of the system, the user being prompted by the value impact platform 120 to apply an intervention based on the percentage risk of readmission.

Interventions are of varied types and include but are not limited to medication management, virtual nurse consultations, in-home support services, and mental health assessments. These interventions are not limited to re-admission issues and encompass a range of healthcare needs. The interventions are applied either at a member-level or at a cluster data object level. When applied at a member-level, each member receives a personalized recommended intervention based on their medical history, risk factors, and other variables such as geographic location or distance to hospital. The intervention is applied as a flag to the entity data object. When applied at a cluster data object level, all members of the particular cluster receive a common set of interventions optimized for that cluster's average or median characteristics.

The generation of interventions also incorporates an efficiency metric that accounts for the effectiveness of the interventions in reducing unnecessary resource utilizations, such as readmissions. This efficiency metric is quantified in terms of reduction in readmissions, and is often balanced against the cost of the intervention to ensure that the overall healthcare system achieves cost savings.

In some embodiments, the success of one or more interventions is tracked by the value impact platform 120. Tracking involves the monitoring and recording of performance indicators such as readmission rate, patient satisfaction, and overall healthcare cost reduction. The collected data is subsequently used to refine the healthcare management model 127 for future scenario modeling predictions. Specifically, the realized success rates of the interventions are incorporated into the model's underlying algorithms, enabling the model to adapt and improve its accuracy in generating subsequent interventions. The ongoing integration of real-world performance data thus contributes to the continual calibration of the healthcare management model 127, thereby facilitating more precise and efficient allocation of healthcare resources and better targeting of alternative care pathways.

In some embodiments, the value impact platform 120 employs a scenario modeling technique to determine one or more possible effects of a determined intervention action on internal system utilization or intervention efficacy. The scenario modeling technique generates one or more scenario model data objects. The scenario model data object is structured to encapsulate distinct recommended focus areas, for instance, specified interventions that propel the overall member population toward particular population states. These population states are selected for their alignment with defined objectives that are stored within the scenario model data object and/or within value impact platform 120. The objectives include, but are not limited to, precise metrics such as the medication adherence, optimization of resource allocation, quantifiable reduction in readmission rates, measurable changes in patient health outcomes, and minimization of healthcare-related expenditures.

The generation of these scenario model data objects is facilitated by the value impact platform 120, through the data processing module 124 and the healthcare management module 126. By leveraging data from health data 110 and other relevant sources, the scenario modeling system analyzes, evaluates, and predicts the potential impact of specific interventions or changes within the network environment 100.

In some embodiments, scenario modeling is executed by assigning one or more weight values to one or more metrics or outcomes associated with one or more of the data sets, to generate an optimized strategy for the healthcare system. These metrics or outcomes include, in some embodiments, a first metric such as the rate of resource utilization, a second metric such as patient readmission rates, and further metrics pertaining to patient care outcomes and cost efficiency as described herein. Each metric is attributed a specific weight that reflects its relative importance or anticipated influence on the system's overarching aims. The assignment of these weights may be initially established based on empirical healthcare data, benchmarks prevalent within the healthcare industry, or the expertise of healthcare practitioners or system administrators. Furthermore, the scenario modeling system is configured to recalibrate these weights automatically in response to shifts in population health trends or modifications in healthcare delivery contracts, such as by applying a goal-seeking algorithm and iteratively modeling varying intervention scenarios. Alternatively, the system allows for manual adjustment of these weight values by authorized users, thereby providing a dual mechanism for dynamic weight adjustment.

The weighting of metrics or outcomes allows the scenario modeling system to balance multiple considerations, such as clinical effectiveness, cost-efficiency, patient satisfaction, and regulatory compliance. For example, if the healthcare system aims to reduce unnecessary readmissions visits while maintaining a high level of patient satisfaction, the scenario modeling system can adjust the weights assigned to these outcomes to find a suitable balance of utilization reduction and alternative care pathway adoption, which in some embodiments would signify patient satisfaction with their medical care.

The user interface module 128 provides a comprehensive visualization of the scenario model data object. It allows users, such as healthcare professionals or administrators, to interact with the data, modify parameters or assumptions, and view updated projections in real-time. This interaction enables the identification of strategies that can drive desired outcomes and optimize the healthcare system's overall performance.

Once the weights are assigned, the scenario modeling system utilizes the healthcare management model 127, which encompasses various algorithms or machine-learning models, to analyze the data and generate predictions. The system considers the relationships between different variables, the potential impact of interventions, and the feasibility of achieving desired outcomes based on the current state of the healthcare system.

Additionally, the scenario modeling system enables users to simulate various scenarios by adjusting the weights of metrics or outcomes, altering assumptions, or modifying input data. This flexibility allows for a thorough exploration of different strategies and their potential outcomes, helping decision-makers to make informed choices that align with the healthcare system's objectives. Furthermore, the scenario modeling system incorporates feedback loops for continuous improvement. As real-world data is collected and analyzed, the system refines its models and adjusts the weights of metrics or outcomes to reflect the most current and accurate information. Furthermore, the scenario modeling system can consider external factors, such as changing regulatory requirements, socio-economic conditions, or advancements in medical technology. This ensures that the system remains adaptive and forward-looking, aligning with the evolving needs of the network environment 100 and the members.

In some embodiments, the value impact platform 120 performs model monitoring. Model monitoring includes assessment one or more model performance metrics and detecting drift in the change in the statistical properties of the data that was used to train the data. In some embodiments, the drift is associated with the interventions impact on the population, as the interventions prove successful the resulting member population metrics will, in some embodiments, drift from the metrics of the starting population. This drift, in some embodiments, is detected as new health data is populated into the system. The value impact platform 120 tracks initial parameters and/or metrics associated with the member data object and identifies changes and/or differences in those parameters over time as new data is populated into the system.

In some embodiments, the value impact platform 120 includes one or more correction mechanisms in response to the detected drift, aiming to adjust and optimize the model for altered data patterns and distributions. These correction mechanisms involve adaptive algorithms that modify model parameters, weight adjustments, or feature recalibration, ensuring that the model remains aligned with the evolving nature of the input data. In certain embodiments, the correction mechanisms employ techniques such as reinforcement learning, transfer learning, or online learning to swiftly adapt to the changing data landscape. Furthermore, these mechanisms might trigger model retraining processes, wherein new data is utilized to update the model, thereby enhancing its predictive accuracy and reliability. In other embodiments, when significant drift is detected, the correction mechanisms might recommend a comprehensive overhaul of the model, encompassing the incorporation of novel features, adjustment of hyperparameters, or even the selection of an alternative modeling approach, thereby maintaining the model's efficacy in dynamically changing environments.

In some embodiments, the value impact platform 120 includes a fairness monitoring to ensure equitable model performance across diverse populations. The value impact platform 120 systematically compares selection rates between predicted outcomes and training data, focusing on attributes including but not limited to gender, age, Area Deprivation Index (ADI) codes, Rural-Urban Commuting Area (RUCA) codes, and Social Determinants of Health (SDOH) Socioeconomic Status (SES) metrics. The fairness monitoring process identifies and mitigate biases, ensuring that the model's predictions do not disproportionately favor or disadvantage any group based on these sensitive features. In some embodiments, the value impact platform 120 includes one or more correction mechanisms in response to fairness modeling. In some embodiments, upon detection of bias or drift through the fairness monitoring component, the value impact platform 120 initiates corrective measures to adjust the model. These measures may include retraining the model with augmented datasets, applying algorithmic fairness techniques, or adjusting predictive thresholds. The platform is configured to automatically implement such corrections to ensure that model outputs remain in alignment with one or more predefined fairness criteria, such as equal opportunity, demographic parity, or the like.

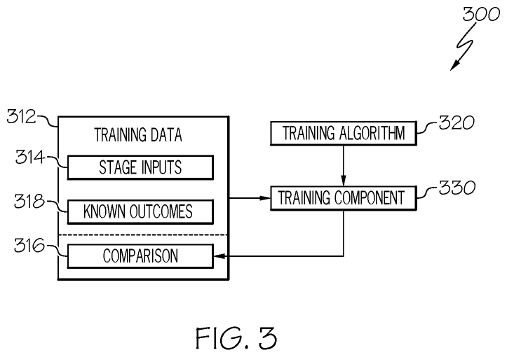

One or more implementations disclosed herein include and/or are implemented using a machine-learning model. For example, one or more of the modules of the value impact platform are implemented using a machine-learning model and/or are used to train the machine-learning model. FIG. 3 shows an example machine-learning training flow chart, according to some embodiments of the disclosure. Referring to FIG. 3, a given machine-learning model is trained using the training flow chart 300. The training data 312 includes one or more of stage inputs 314 and the known outcomes 318 related to the machine-learning model to be trained. The stage inputs 314 are from any applicable source including text, visual representations, data, values, comparisons, and stage outputs, e.g., one or more outputs from one or more steps from FIG. 2. The known outcomes 318 are included for the machine-learning models generated based on supervised or semi-supervised training, or can based on known labels, such as topic labels. An unsupervised machine-learning model is not trained using the known outcomes 318. The known outcomes 318 includes known or desired outputs for future inputs similar to or in the same category as the stage inputs 314 that do not have corresponding known outputs.

The training data 312 and a training algorithm 320, e.g., one or more of the modules implemented using the machine-learning model and/or are used to train the machine-learning model, is provided to a training component 330 that applies the training data 312 to the training algorithm 320 to generate the machine-learning model. According to an implementation, the training component 330 is provided comparison results 316 that compare a previous output of the corresponding machine-learning model to apply the previous result to re-train the machine-learning model. The comparison results 316 are used by the training component 330 to update the corresponding machine-learning model. The training algorithm 320 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIG. 2 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

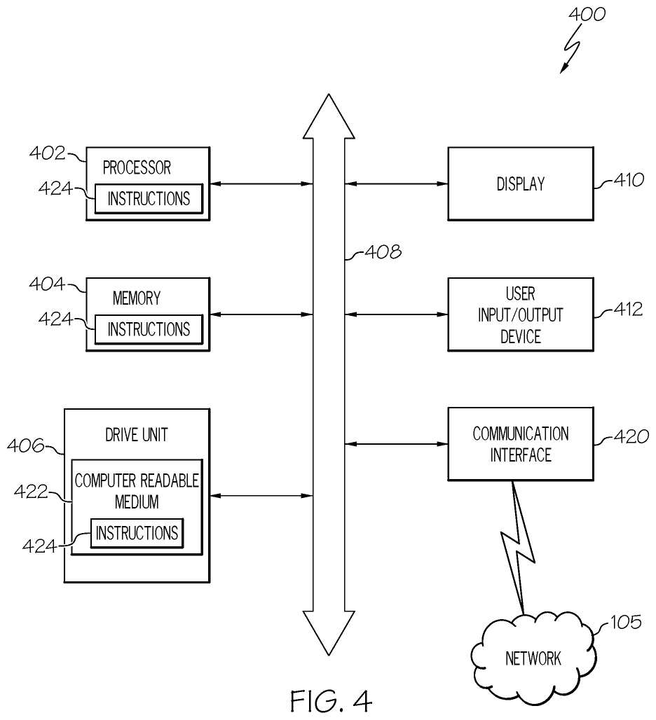

FIG. 4 illustrates an implementation of a computer system that executes techniques presented herein. The computer system 400 includes a set of instructions that are executed to cause the computer system 400 to perform any one or more of the methods or computer based functions disclosed herein. The computer system 400 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 400 operates in the capacity of a server or as a client user computer in a server-client user network environment, or as a peer computer system in a peer-to-peer (or distributed) network environment. The computer system 400 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 400 is implemented using electronic devices that provide voice, video, or data communication. Further, while the computer system 400 is illustrated as a single system, the term “system” shall also be taken to include any collection of systems or sub-systems that individually or jointly execute a set, or multiple sets, of instructions to perform one or more computer functions.

As illustrated in FIG. 4, the computer system 400 includes a processor 402, e.g., a central processing unit (CPU), a graphics processing unit (GPU), or both. The processor 402 is a component in a variety of systems. For example, the processor 402 is part of a standard personal computer or a workstation. The processor 402 is one or more processors, digital signal processors, application specific integrated circuits, field programmable gate arrays, servers, networks, digital circuits, analog circuits, combinations thereof, or other now known or later developed devices for analyzing and processing data. The processor 402 implements a software program, such as code generated manually (i.e., programmed).

The computer system 400 includes a memory 404 that communicates via bus 408. The memory 404 is a main memory, a static memory, or a dynamic memory. The memory 404 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 404 includes a cache or random-access memory for the processor 402. In alternative implementations, the memory 404 is separate from the processor 402, such as a cache memory of a processor, the system memory, or other memory. The memory 404 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts, or tasks illustrated in the figures or described herein are performed by the processor 402 executing the instructions stored in the memory 404. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 400 further includes a display 410, such as a liquid crystal display (LCD), an organic light emitting diode (OLED), a flat panel display, a solid-state display, a cathode ray tube (CRT), a projector, a printer or other now known or later developed display device for outputting determined information. The display 410 acts as an interface for the user to see the functioning of the processor 402, or specifically as an interface with the software stored in the memory 404 or in the drive unit 406.

Additionally or alternatively, the computer system 400 includes an input/output device 412 configured to allow a user to interact with any of the components of the computer system 400. The input/output device 412 is a number pad, a keyboard, a cursor control device, such as a mouse, a joystick, touch screen display, remote control, or any other device operative to interact with the computer system 400.

The computer system 400 also includes the drive unit 406 implemented as a disk or optical drive. The drive unit 406 includes a computer-readable medium 422 in which one or more sets of instructions 424, e.g. software, is embedded. Further, the sets of instructions 424 embodies one or more of the methods or logic as described herein. The sets of instructions 424 resides completely or partially within the memory 404 and/or within the processor 402 during execution by the computer system 400. The memory 404 and the processor 402 also include computer-readable media as discussed above.

In some systems, computer-readable medium 422 includes the set of instructions 424 or receives and executes the set of instructions 424 responsive to a propagated signal so that a device connected to network 105 communicates voice, video, audio, images, or any other data over the network 105. Further, the sets of instructions 424 are transmitted or received over the network 105 via the communication port or interface 420, and/or using the bus 408. The communication port or interface 420 is a part of the processor 402 or is a separate component. The communication port or interface 420 is created in software or is a physical connection in hardware. The communication port or interface 420 is configured to connect with the network 105, external media, the display 410, or any other components in the computer system 400, or combinations thereof. The connection with the network 105 is a physical connection, such as a wired Ethernet connection, or is established wirelessly as discussed below. Likewise, the additional connections with other components of the computer system 400 are physical connections or are established wirelessly. The network 105 alternatively be directly connected to the bus 408.

While the computer-readable medium 422 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 422 is non-transitory, and may be tangible.

The computer-readable medium 422 includes a solid-state memory such as a memory card or other package that houses one or more non-volatile read-only memories. The computer-readable medium 422 is a random-access memory or other volatile re-writable memory. Additionally or alternatively, the computer-readable medium 422 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 400 is connected to the network 105. The network 105 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 105 includes wide area networks (WAN), such as the Internet, local area networks (LAN), campus area networks, metropolitan area networks, a direct connection such as through a Universal Serial Bus (USB) port, or any other networks that allows for data communication. The network 105 is configured to couple one computing device to another computing device to enable communication of data between the devices. The network 105 is generally enabled to employ any form of machine-readable media for communicating information from one device to another. The network 105 includes communication methods by which information travels between computing devices. The network 105 is divided into sub-networks. The sub-networks allow access to all of the other components connected thereto or the sub-networks restrict access between the components. The network 105 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the disclosure.

In the description provided herein, numerous specific details are set forth. However, it is understood that embodiments of the disclosure are practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognize that other and further modifications are made thereto without departing from the spirit of the disclosure, and it is intended to claim all such changes and modifications as falling within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects:

Example 1. A computer-implemented method, the method comprising: receiving, by one or more processors, a first data object, the first data object including: an entity data set containing a plurality of entities; a first data set including request data associated with the plurality of entities; an event data set; and a plurality of data sets associated with one or more performance metrics; generating, by the one or more processors, based on at least one of the entity data set, the first data set, or the event data set, an entity data object for each of the plurality of entities; applying, by the one or more processors, a machine-learning model to the entity data objects generated for the plurality of entities, the machine-learning model trained to identify a correlation between the entity data object for each of the plurality of entities and a probability of re-utilization of one or more resources; determining, by the one or more processors, based on the application of the machine-learning model to the entity data objects, a prediction indicator for each entity of the plurality of entities; generating, by the one or more processors, a re-utilization offset data object for each of the plurality of entities, the re-utilization offset data object based on the prediction indicator determined for the entity; and causing, by the one or more processors, one or more of the re-utilization offset data objects generated for the plurality of entities to be displayed on a Graphical User Interface (GUI).

Example 2. The computer-implemented method of Example 1, further comprising: for each entity, assigning an intervention flag based on the entity data object and the prediction indicator determined for the entity.

Example 3. The computer-implemented method of Example 2, wherein the plurality of data sets associated with the one or more performance metrics includes a determinate data set, and wherein assigning the intervention flag is further based on the determinate data set.

Example 4. The computer-implemented method of Example 2, wherein the intervention flag includes instruction data, the instruction data being associated with one or more management pathways for the respective entity.

Example 5. The computer-implemented method of any of Examples 1-4, wherein the prediction indicator is a numeric score, the score indicative of a likelihood the respective entity re-utilizes a resource during a pre-determined time period.

Example 6. The computer-implement method of any of Examples 1-5, wherein the event data set comprises one or more of an episode treatment groupers array, a service categories array, or a data records array associated with admissions, discharges, and transfers.

Example 7. The computer-implemented method of any of Examples 1-6, wherein the re-utilization offset data object generated for each entity is further based on a reduction of total resource utilization associated with a management pathway.

Example 8. The computer-implemented method of Example 7, wherein the re-utilization offset data object is further based on a likelihood that an implementation of the management pathway avoids a utilization of one or more resources.

Example 9. The computer-implemented method of any of Examples 1-8, wherein the entity data object is generated for each member during a specific phase of an admission cycle, the specific phase selected from a group consisting of: admission, transfer, and discharge, and wherein the entity data object is updated with new data received about the member at each respective phase.

Example 10. A system comprising memory and one or more processors communicatively coupled to the memory, the one or more processors configured to: receive, a first data object, the first data object including: an entity data set containing a plurality of entities; a first data set including request data associated with the plurality of entities; an event data set; and a plurality of data sets associated with one or more performance metrics; generate, based on at least one of the entity data set, the first data set, or the event data set, an entity data object for each of the plurality of entities; apply a machine-learning model to the entity data objects generated for the plurality of entities, the machine-learning model trained to identify a correlation between the entity data object for each of the plurality of entities and a probability of re-utilization of one or more resources; determine, based on the application of the machine-learning model to the entity data objects, a prediction indicator for each entity of the plurality of entities; generate, a re-utilization offset data object for each of the plurality of entities, the re-utilization offset data object based on the prediction indicator determined for the entity; and cause one or more of the re-utilization offset data objects generated for the plurality of entities to be displayed on a Graphical User Interface (GUI).

Example 11. The system of Example 10, the one or more processors further configured to: for each entity, assign an intervention flag based on the entity data object and the prediction indicator determined for the entity.

Example 12. The system of Example 11, wherein the plurality of data sets associated with the one or more performance metrics includes a determinate data set, and wherein assigning the intervention flag is further based on the determinate data set.

Example 13. The system of Example 11, wherein the intervention flag includes instruction data, the instruction data being associated with one or more management pathways for the respective entity.

Example 14. The system of any of Examples 10-13, wherein the prediction indicator is a numeric score, the score indicative of a likelihood the respective entity re-utilizes a resource during a pre-determined time period.

Example 15. The system of any of Examples 10-14, wherein the event data set comprises one or more of an episode treatment groupers array, a service categories array, or a data records array associated with admissions, discharges, and transfers.

Example 16. The system of any of Examples 10-15, wherein the re-utilization offset data object generated for each entity is based on a reduction of total resource utilization associated with a management pathway.

Example 17. The system of Example 16, wherein the re-utilization offset data object is further based on a likelihood that an implementation of the management pathway avoids a utilization of one or more resources.

Example 18. The system of any of Examples 10-18, wherein the entity data object is generated for each member during a specific phase of an admission cycle, the specific phase selected from a group consisting of: admission, transfer, and discharge, and wherein the entity data object is updated with new data received about the member at each respective phase.

Example 19. One or more non-transitory computer-readable storage media including instructions that, when executed by one or more processors, cause the one or more processors to: receive, a first data object, the first data object including: an entity data set containing a plurality of entities; a first data set including request data associated with the plurality of entities; an event data set; and a plurality of data sets associated with one or more performance metrics; generate, based on at least one of the entity data set, the first data set, or the event data set, an entity data object for each of the plurality of entities; apply a machine-learning model to the entity data objects generated for the plurality of entities, the machine-learning model trained to identify a correlation between the entity data object for each of the plurality of entities and a probability of re-utilization of one or more resources; determine, based on the application of the machine-learning model to the entity data objects, a prediction indicator for each entity of the plurality of entities; assign, for each entity of the entity data object, an intervention flag based on the entity data object and the prediction indicator determined for the entity, the intervention flag including a management pathway; generate, a re-utilization offset data object for each of the plurality of entities, the re-utilization offset data object based on the prediction indicator determined for the entity, wherein the re-utilization offset data object includes information related to a total resource utilization associated with an intervention flag; and cause one or more of the re-utilization offset data objects generated for the plurality of entities to be displayed on a Graphical User Interface (GUI).

Example 20. The one or more non-transitory computer-readable storage media of Example 19, wherein the re-utilization offset data object is further based on a likelihood that an implementation of the management pathway avoids a utilization of one or more resources.

What is claimed is:

1. A computer-implemented method, the method comprising:
receiving, by one or more processors, a first data object, the first data object including:
an entity data set containing a plurality of entities;
a first data set including request data associated with the plurality of entities;
an event data set; and
a plurality of data sets associated with one or more performance metrics;
generating, by the one or more processors, based on at least one of the entity data set, the first data set, or the event data set, an entity data object for each of the plurality of entities;
applying, by the one or more processors, a machine-learning model to the entity data objects generated for the plurality of entities, the machine-learning model trained to identify a correlation between the entity data object for each of the plurality of entities and a probability of re-utilization of one or more resources;
determining, by the one or more processors, based on the application of the machine-learning model to the entity data objects, a prediction indicator for each entity of the plurality of entities;
generating, by the one or more processors, a re-utilization offset data object for each of the plurality of entities, the re-utilization offset data object based on the prediction indicator determined for the entity; and
causing, by the one or more processors, one or more of the re-utilization offset data objects generated for the plurality of entities to be displayed on a Graphical User Interface (GUI).

2. The computer-implemented method of claim 1, further comprising: for each entity, assigning an intervention flag based on the entity data object and the prediction indicator determined for the entity.

3. The computer-implemented method of claim 2, wherein the plurality of data sets associated with the one or more performance metrics includes a determinate data set, and wherein assigning the intervention flag is further based on the determinate data set.

4. The computer-implemented method of claim 2, wherein the intervention flag includes instruction data, the instruction data being associated with one or more management pathways for the respective entity.

5. The computer-implemented method of claim 1, wherein the prediction indicator is a numeric score, the score indicative of a likelihood the respective entity re-utilizes a resource during a pre-determined time period.

6. The computer-implement method of claim 1, wherein the event data set comprises one or more of an episode treatment groupers array, a service categories array, or a data records array associated with admissions, discharges, and transfers.

7. The computer-implemented method of claim 1, wherein the re-utilization offset data object generated for each entity is further based on a reduction of total resource utilization associated with a management pathway.

8. The computer-implemented method of claim 7, wherein the re-utilization offset data object is further based on a likelihood that an implementation of the management pathway avoids a utilization of one or more resources.

9. The computer-implemented method of claim 1, wherein the entity data object is generated for each member during a specific phase of an admission cycle, the specific phase selected from a group consisting of: admission, transfer, and discharge, and wherein the entity data object is updated with new data received about the member at each respective phase.

10. A system comprising memory and one or more processors communicatively coupled to the memory, the one or more processors configured to:
receive, a first data object, the first data object including:
an entity data set containing a plurality of entities;
a first data set including request data associated with the plurality of entities;
an event data set; and
a plurality of data sets associated with one or more performance metrics;
generate, based on at least one of the entity data set, the first data set, or the event data set, an entity data object for each of the plurality of entities;
apply a machine-learning model to the entity data objects generated for the plurality of entities, the machine-learning model trained to identify a correlation between the entity data object for each of the plurality of entities and a probability of re-utilization of one or more resources;
determine, based on the application of the machine-learning model to the entity data objects, a prediction indicator for each entity of the plurality of entities;
generate, a re-utilization offset data object for each of the plurality of entities, the re-utilization offset data object based on the prediction indicator determined for the entity; and
cause one or more of the re-utilization offset data objects generated for the plurality of entities to be displayed on a Graphical User Interface (GUI).

11. The system of claim 10, the one or more processors further configured to: for each entity, assign an intervention flag based on the entity data object and the prediction indicator determined for the entity.

12. The system of claim 11, wherein the plurality of data sets associated with the one or more performance metrics includes a determinate data set, and wherein assigning the intervention flag is further based on the determinate data set.

13. The system of claim 11, wherein the intervention flag includes instruction data, the instruction data being associated with one or more management pathways for the respective entity.

14. The system of claim 10, wherein the prediction indicator is a numeric score, the score indicative of a likelihood the respective entity re-utilizes a resource during a pre-determined time period.

15. The system of claim 10, wherein the event data set comprises one or more of an episode treatment groupers array, a service categories array, or a data records array associated with admissions, discharges, and transfers.

16. The system of claim 10, wherein the re-utilization offset data object generated for each entity is based on a reduction of total resource utilization associated with a management pathway.

17. The system of claim 16, wherein the re-utilization offset data object is further based on a likelihood that an implementation of the management pathway avoids a utilization of one or more resources.

18. The system of claim 10, wherein the entity data object is generated for each member during a specific phase of an admission cycle, the specific phase selected from a group consisting of: admission, transfer, and discharge, and wherein the entity data object is updated with new data received about the member at each respective phase.

19. One or more non-transitory computer-readable storage media including instructions that, when executed by one or more processors, cause the one or more processors to:
receive, a first data object, the first data object including:
an entity data set containing a plurality of entities;
a first data set including request data associated with the plurality of entities;
an event data set; and
a plurality of data sets associated with one or more performance metrics;
generate, based on at least one of the entity data set, the first data set, or the event data set, an entity data object for each of the plurality of entities;
apply a machine-learning model to the entity data objects generated for the plurality of entities, the machine-learning model trained to identify a correlation between the entity data object for each of the plurality of entities and a probability of re-utilization of one or more resources;
determine, based on the application of the machine-learning model to the entity data objects, a prediction indicator for each entity of the plurality of entities;
assign, for each entity of the entity data object, an intervention flag based on the entity data object and the prediction indicator determined for the entity, the intervention flag including a management pathway;
generate, a re-utilization offset data object for each of the plurality of entities, the re-utilization offset data object based on the prediction indicator determined for the entity, wherein the re-utilization offset data object includes information related to a total resource utilization associated with an intervention flag; and
cause one or more of the re-utilization offset data objects generated for the plurality of entities to be displayed on a Graphical User Interface (GUI).

20. The one or more non-transitory computer-readable storage media of claim 19, wherein the re-utilization offset data object is further based on a likelihood that an implementation of the management pathway avoids a utilization of one or more resources.
